(19) United States
(12) Patent Application Publication
(10) Pub. No.: US 2023/0101946 A1
(43) Pub. Date: Mar. 30, 2023

(54) SYSTEM AND METHOD FOR INTELLIGENT DIAGNOSIS

(71) Applicant: Dr. Dadjou Dentistry Professional Corporation, Mississauga (CA)

(72) Inventor: Hamed DADJOU, Mississauga (CA)

(21) Appl. No.: 17/956,012

(22) Filed: Sep. 29, 2022

Related U.S. Application Data
(60) Provisional application No. 63/249,835, filed on Sep. 29, 2021.

(51) Int. Cl. G16H 50/20; G16H 40/20; G16H 40/67; A61C 9/00; G06T 7/00; G01N 33/00

(52) U.S. Cl. CPC G16H 50/20; G16H 40/20; G16H 40/67; A61C 9/0053; G06T 7/0012; G01N 33/0075; G06T 2207/30036

(57) ABSTRACT

A computer system and method for intelligent diagnosis are provided. The system comprises at least one processor and a memory storing instructions which when executed by the processor configure the processor to perform the method. The method comprises receiving an initial appoint request, receiving a first scanning appointment request, sending a movable practice to patient location, receiving scanning data, and sending the scanning data to remote server for processing.

### CROSS REFERENCE TO RELATED APPLICATIONS

This application is a non-provisional of, and claims all benefit, including priority to U.S. Application No. 63/249,835, dated 29 Sep. 2021, entitled SYSTEM AND METHOD FOR INTELLIGENT DIAGNOSIS, incorporated herein in its entirety by reference.

### FIELD

The present disclosure generally relates to diagnosis, and in particular to a system and method for intelligent diagnosis.

### INTRODUCTION

Many patients are reluctant to go to the dental office due to availability and time schedule. Some patients may suffer trauma in the idea of going to a dental office (e.g., past experiences on procedure and doctor). Patients may dislike waiting in waiting rooms and may not like sounds they may hear. Some patients think going to the dentist's office is a waste of time. Some patients with some disabilities are not likely to able to go to the office and access treatment easily. Some patients might not be able to get their preferred appointment time.

### SUMMARY

In accordance with an aspect, there is provided a computer system for intelligent diagnosis. The system comprises at least one processor and a memory storing instructions which when executed by the processor configure the processor to receive an initial appoint request, receive a first scanning appointment request, send a movable practice to patient location, receive scanning data, and send the scanning data to remote server for processing.

In accordance with another aspect, there is provided a computer-implemented method for intelligent diagnosis. The method comprises receiving an initial appoint request, receiving a first scanning appointment request, sending a movable practice to patient location, receiving scanning data, and sending the scanning data to remote server for processing.

In accordance with another aspect, there is provided a system for intelligent diagnosis using a scanning device. The system for intelligent diagnosis comprises a scan unit having a camera server and a background tasks manager, one or more application server in communication with the scan unit, a cloud computing server connected to the scan unit and the data server, and one or more data servers connected to the scan unit and to the cloud computing server. The one or more data servers are configured to send and store data to and from the scan unit and the cloud computing server. The cloud computer server is configured to use one or more processing modules.

In one embodiment, the cloud computing server comprises a model management server and a training server.

In another embodiment, the cloud computing server further comprises a teeth detection module, and a teeth recognition module.

In yet another embodiment, the cloud computing server can identify an error in one or more processing modules and replace the said processing module with a replica that has the same functions of the said processing module.

The cloud computing server, in another embodiment, further comprises an audio/video analyzer module, sound recognition module, speaker recognition module, context maker module, command queue manager module, command runner module.

The training server, in another embodiment, comprises an automatic speech recognition (ASR) module, natural language processing (NLP) module, text-to-speech synthesis (TTS) module, teeth detection module, and teeth recognition module.

The cloud computing server, in still another embodiment, further comprises a video core feature module, skills module, and contribution module.

It is also an object of the present invention to provide a scanning device for examining an oral cavity.

In one embodiment, the scanning device further comprises a data processing unit coupled to the data acquisition module and configured to operate the data acquisition module.

In another embodiment, the scanning device may further comprise a power management module for regulating the supplied power to the main processing unit and the data acquisition module.

In yet another embodiment, the scanning device acquires at least one oral feature via the data acquisition module, wherein the oral feature can be impressions of the teeth, gingiva, oral soft tissues, bite relationships, tongue, surfaces of the oral cavity, or combinations thereof.

In still another embodiment, the scanning device may further comprise a communications module for sending and receiving data from another scanning device or a remote processing device.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates an example of a basic model of an intelligent system;

FIG. 2 illustrates, in a graph, an example of a schematic construction of an ANN, in accordance with some embodiments;

FIG. 3 illustrates an example of a parallel system architecture, in accordance with some embodiments;

FIG. 4 illustrate an example of an ANN model of parallel system, in accordance with some embodiments;

FIGS. 5 and 6 illustrate examples of diagrams of cascaded systems, in accordance with some embodiments;

FIG. 7 illustrates an example of a model of a diagnosis AI, in accordance with some embodiments;

FIG. 8 illustrates a model of a triage AI, in accordance with some embodiments;

FIG. 9 illustrates a model of an electric vehicle AI, in accordance with some embodiments;

FIG. 10 illustrates input and output parameters of the electric vehicle AI, in accordance with some embodiments;

FIG. 11, illustrates, an example of an AI management system, in accordance with some embodiments;

FIG. 12 illustrates an example of a remote dental clinic, in accordance with some embodiments;

FIG. 13 illustrates, in a flowchart, an example of a method of data acquisition, in accordance with some embodiments;

FIG. 14 illustrates, in a flowchart, an example of a method of diagnosis, in accordance with some embodiments;

FIG. 15 illustrates an example of a model for an AI for diagnosis, in accordance with some embodiments.

FIG. 16 illustrates another example of a model for an AI for diagnosis, in accordance with some embodiments;

FIG. 17 illustrates, in a schematic diagram, an example of a machine learning prediction platform, in accordance with some embodiments;

FIG. 18 is a schematic diagram of a scanning device, in accordance with some embodiments;

FIG. 19 illustrates, in a block diagram, the network connection of the software and processing units of the system, in accordance with some embodiments; and

FIG. 20 is a schematic diagram of a computing device such as a server or other computer in a device.

It is understood that throughout the description and figures, like features are identified by like reference numerals.

### DETAILED DESCRIPTION

Embodiments of methods, systems, and apparatus are described through reference to the drawings. Applicant notes that the described embodiments and examples are illustrative and non-limiting. Practical implementation of the features may incorporate a combination of some or all of the aspects, and features described herein should not be taken as indications of future or existing product plans.

In some embodiments, remote medical analysis, dental examination, and appointment registration systems are provided.

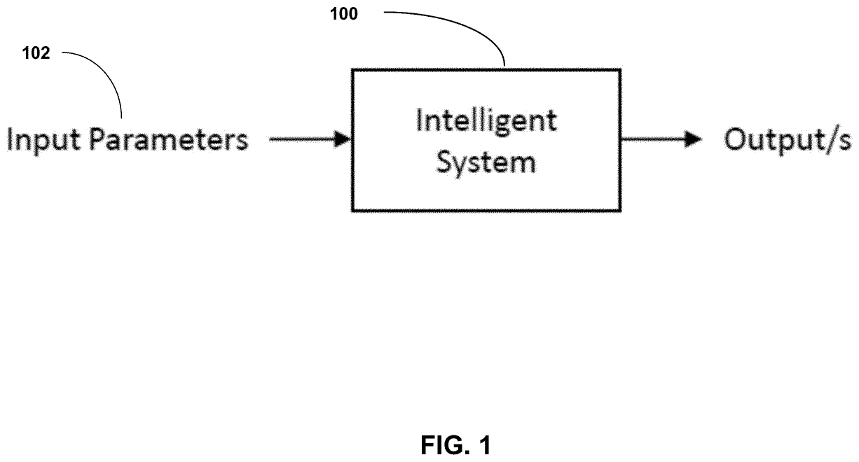

FIG. 1 illustrates an example of a basic model of an intelligent system 100. The intelligent system 100 will process input parameters 102. The input parameters 102 can be anything from patient health records, sensor data, data from the robotic diagnostic tool, data from the patient's wearable device, electric car data, or any other data that we can use. The intelligent system 100 can be any AI algorithm available. In some embodiments an artificial neural network (ANN) will be used ANN is a branch of artificial intelligence and is a computational system that simulate the neurons of a biological nerve system. It is used for information processing to find knowledge, patterns, or models from a large amount of data.

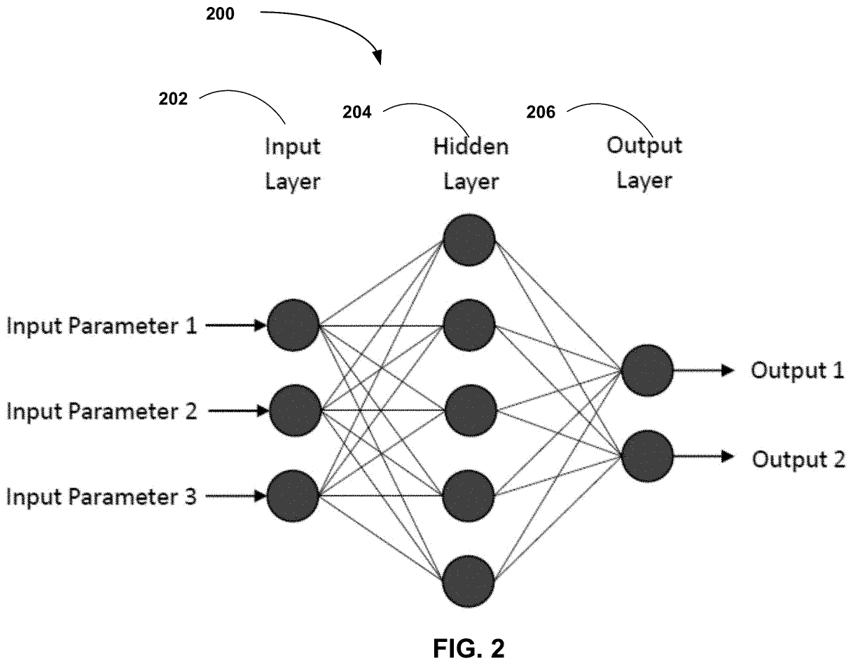

FIG. 2 illustrates, in a graph, an example of a schematic construction 200 of an ANN, in accordance with some embodiments. The input layer 202 receives the input from different sources The hidden layers 204 are the neurons that are connected with each other. These are similar to our brain if simple decisions are made such as determining the shape of an object then our brains use minimal processing power or less neurons as compared to more complex task of diagnosing a patient. The connection between the input and the neurons have some “weight” that determines their strength of connection. These connections can be likened to ropes with varying thicknesses. I.e., it can be simply said that a thread is weaker than a rope. The outputs 206 are the decision that we want the ANN to derive.

In order for the ANN to perform well, it if first trained. For the training, a large number of data is used. For example, hundreds of data points may be used to “teach” the ANN to make a “decision”.

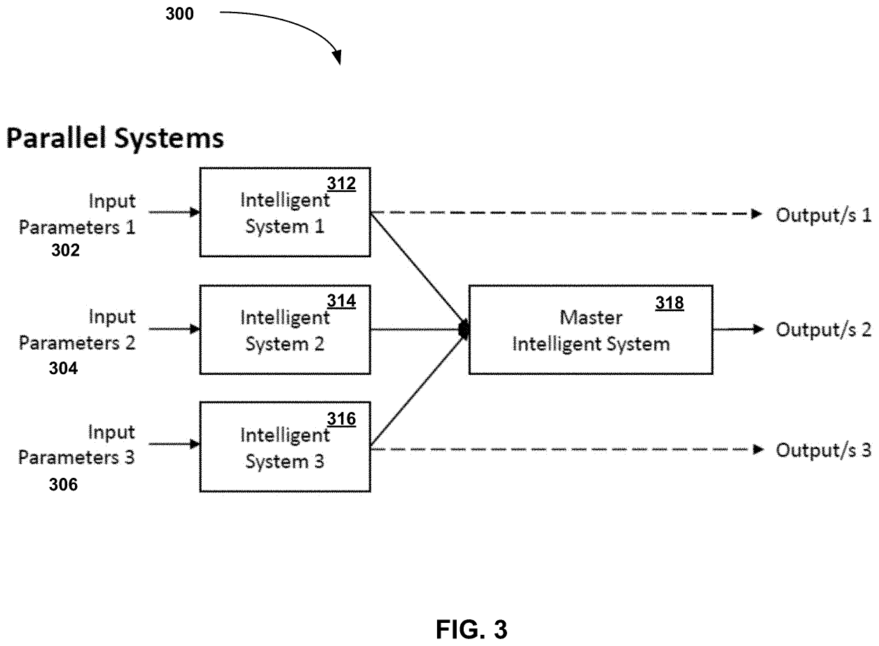

FIG. 3 illustrates an example of a parallel system architecture 300, in accordance with some embodiments. In this example, four intelligent systems are connected with each other. However, it should be understood that any number of intelligent systems may be used. Intelligent systems 1, 2 and 3 (312, 314, 316) may have different input parameters 302, 304, 306 and may come up with at least one output. Another output may be used by a master intelligent system 318. The master intelligent system 318 may require higher processing power than the first three intelligent systems 312, 314, 316. Therefore, this 318 can be installed in a computer or laptop rather than an iPad or a handheld device. In this example, it is imagined that the master intelligent system 318 is located in a remote computing device or cloud service which the electric cars may connect via 5G, for example. However, as can also be seen based on FIG. 3 that master intelligent system 318 is dependent on the output of intelligent systems 1, 2 and 3 (312, 314, 316).

There can be any number of intelligent systems that can be in parallel with each other. The maximum number will depend on the processing capacity of the computer or laptop. The input parameters 302, 304, 306 can be any number of input parameters that are needed to derive an output or to make a decision. For example, to identify a type of fruit, the shape of the fruit, the color, the texture, and the taste may be obtained. However, to determine the health condition of a type of tooth, different and more input parameters may be needed.

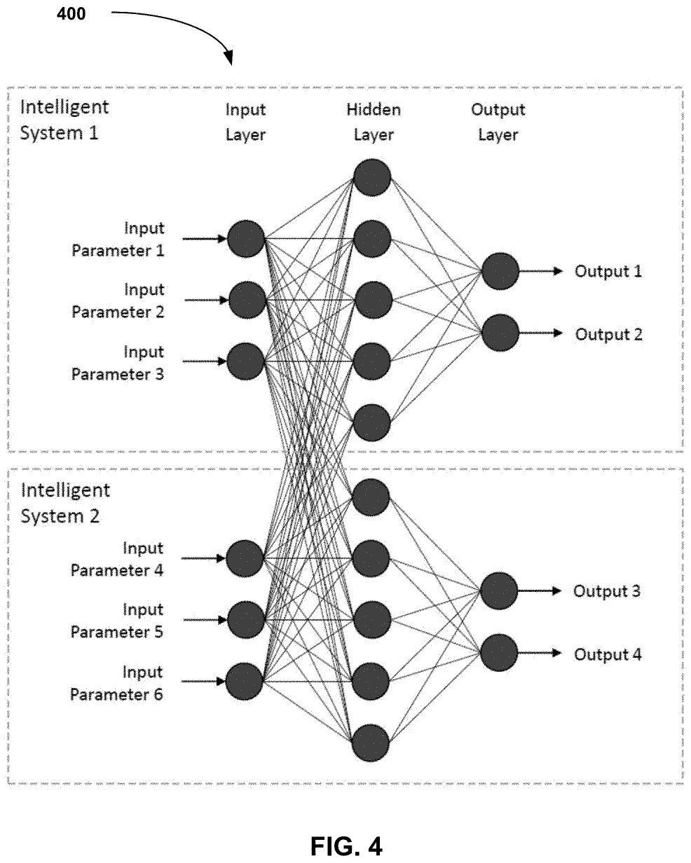

FIG. 4 illustrate an example of an ANN model of parallel system 400, in accordance with some embodiments. FIG. 4 shows an how the artificial neural network may be connected using a parallel structure. Here, there are four outputs that are dependent to input parameters 1 to 6. I.e., a change in input 1 may affect output 5, for example. To design these types of system, data that will most likely affect a particular output may be grouped together. For example, the data derived from a robotic diagnostic device may determine the health condition of the tooth, while an electric car's battery level is not needed to diagnose gingivitis. However, the electric car's battery level may affect the scheduling.

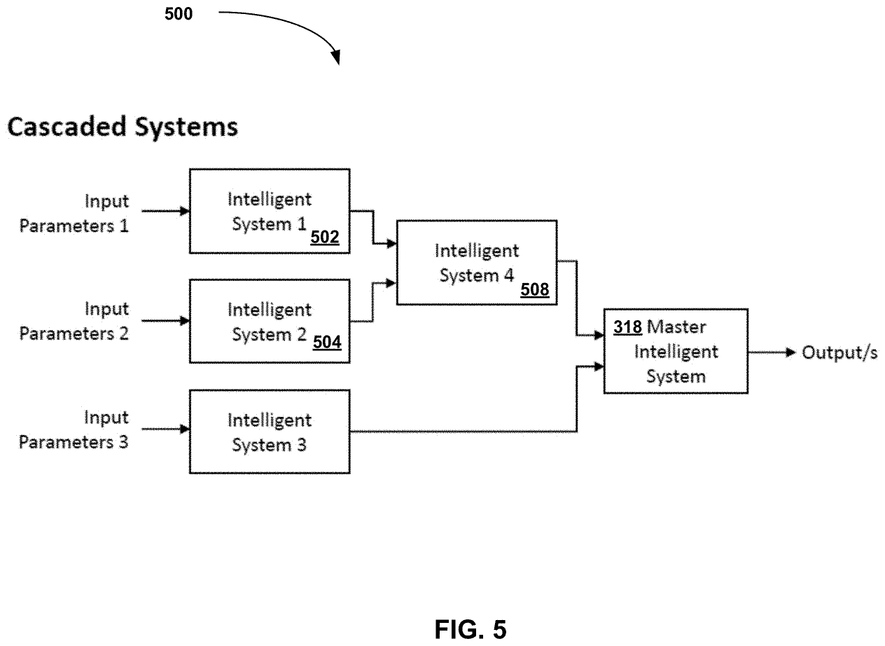

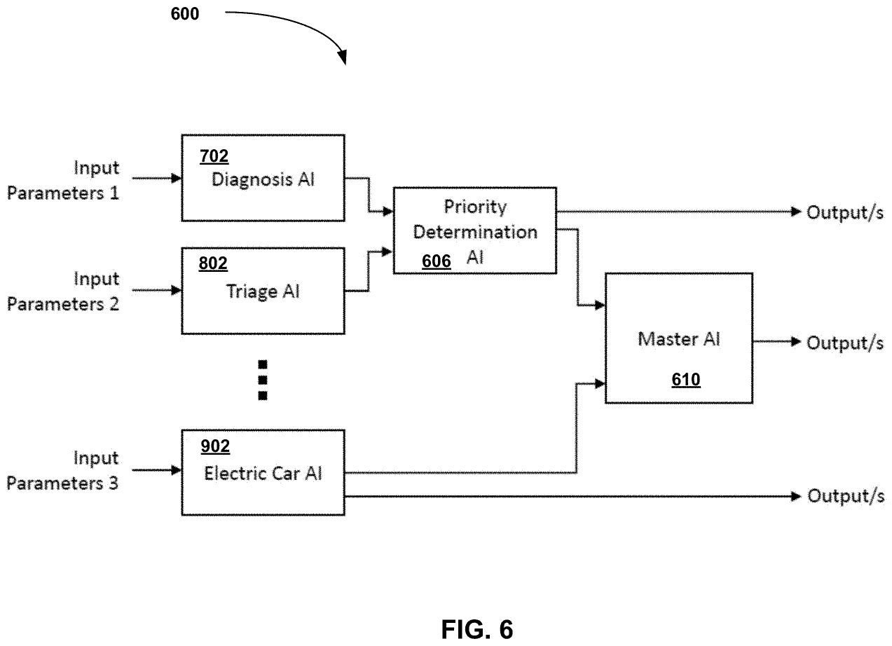

FIGS. 5 and 6 illustrate examples of diagrams of cascaded systems 500, 600, in accordance with some embodiments. The dependency of other intelligent systems to the output of the connected intelligent systems is shown in FIGS. 5 and 6. For example, intelligent system 4 508 needs the output of intelligent systems 1 502 and 2 504. The master intelligent system 318 may need some complex data from the other intelligent systems to come up of at least one output. FIG. 6 shows a more practical example of how the parallel cascaded system combination may be used to make a complex decision. Here, the master AI 610 uses the priority data 606 and the output of the electric car AI 902 in order to derive an efficient scheduling data.

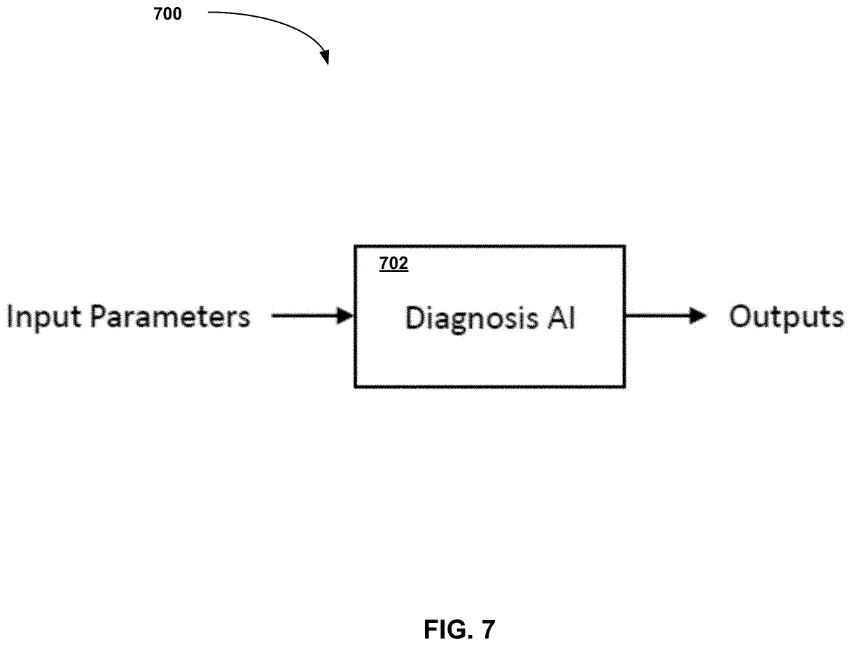

FIG. 7 illustrates an example of a model 700 of a diagnosis AI 702, in accordance with some embodiments. To design each AI system of the intelligent diagnosis system prototype, the basic model may be used. A set of input parameters are defined in order to derive a diagnosis. For example, “What are the indications of gingivitis?” and/or “Where can we get the indications? Via physical examination, image data/color of the gums, etc.?”

A processing method can be a pattern recognition tool. For example, an image of a patient's gum may be obtained using a robotic diagnostic device. The pattern recognition tool can be an image processing system which will compare the image that we got to a “gingivitis image template” that is stored in the AI database. Alternatively, if gingivitis relies mostly on the color of the gums, then the “color values” of the acquired image data may be obtained which can be in the form of RGB or CMYK. If the color values are within the range of gingivitis color indicator, then the AI may decide that there is a high chance of gingivitis.

Another example may comprise the identification of the type of tooth. Here, the input parameters can be the dimensions of the tooth length, width, height, dental position, etc.

The AI can also be used to determine the presence or degree of cavities in a particular tooth or to the whole dental area. The robotic diagnostic tool may identify cavities in the surface, in between teeth, sides peripheries, or even the hidden cavities.

Other input parameters may include light properties that may reflect or penetrate the tooth. This may help in assessing the tooth color in which the AI may suggest a tooth whitening procedure. The output parameters can also be the predicted time to complete the dental procedures, risk assessment based on the patient's underlying conditions, urgency of the procedure, tooth extraction, oral prophylaxis, root canal, or any other dental procedure or dental health score.

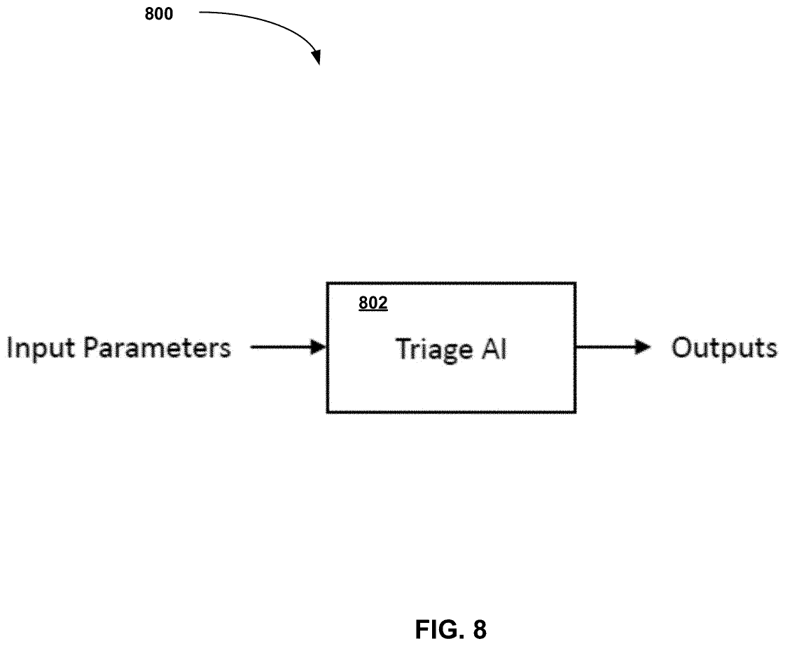

FIG. 8 illustrates a model 800 of a triage AI 802, in accordance with some embodiments. The triage AI 800 may use a different set of input parameters than the diagnosis AI. For example, the following may be obtained: the intensity of pain on a scale of 0 to 10; if the patient is taking any medications (Yes/No); if the medications relieve the pain (Yes/No); and a particular type of pain (a value may be assigned for the particular type of pain which the AI can use). The output then can be the priority level of the patient or an emergency alert, if needed. These outputs can be used by the master AI to determine which electric car to use with adequate equipment to attend to the patient.

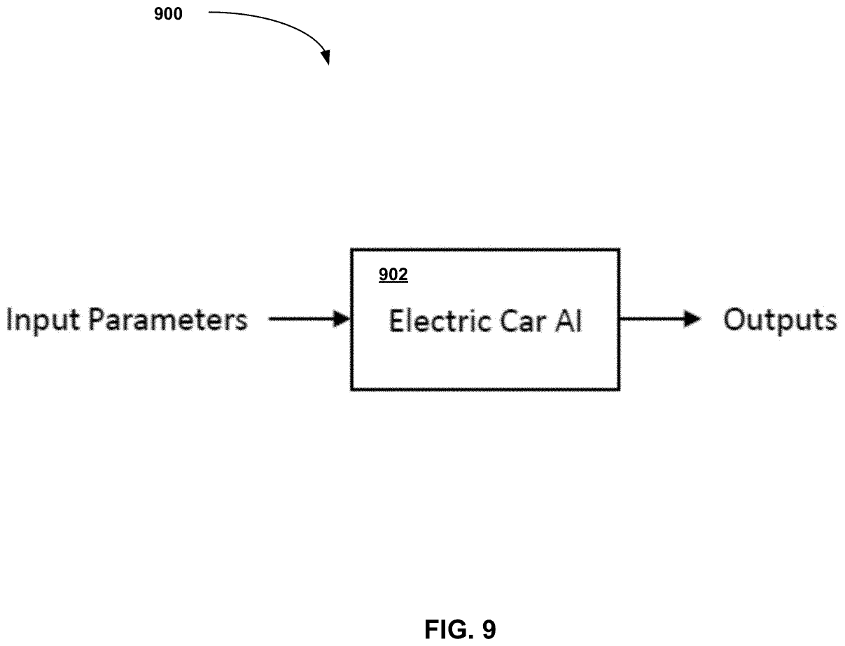

FIG. 9 illustrates a model 900 of an electric vehicle AI 902, in accordance with some embodiments. The basic model for the AI is used.

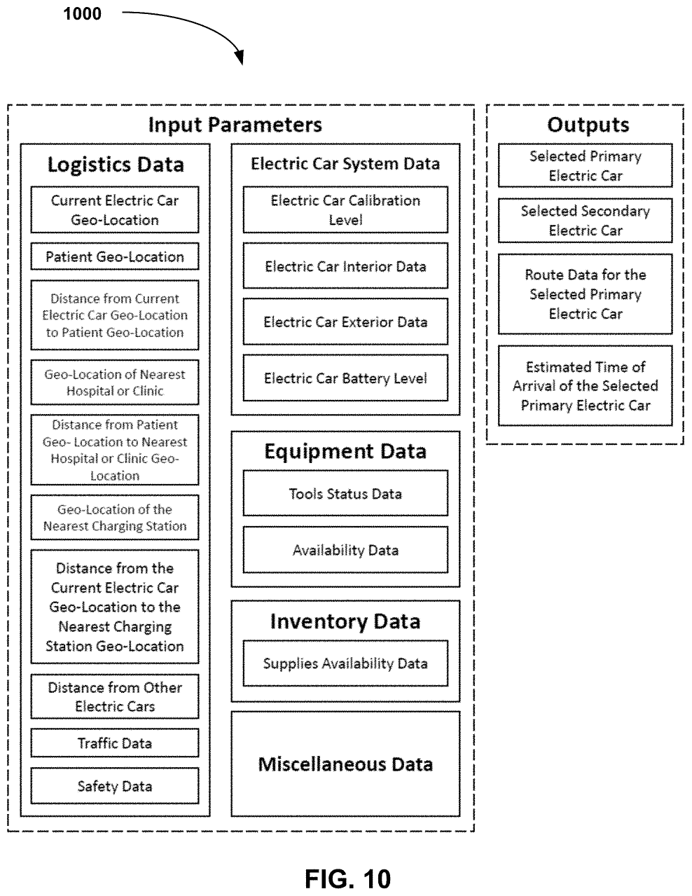

FIG. 10 illustrates input and output parameters 1000 of the electric vehicle AI model 900, in accordance with some embodiments. FIG. 10 shows a non-exhaustive list of the input and output parameters 1000. These parameters 1000 may be used in the scheduling AI or the master AI to determine the priority patients, for example.

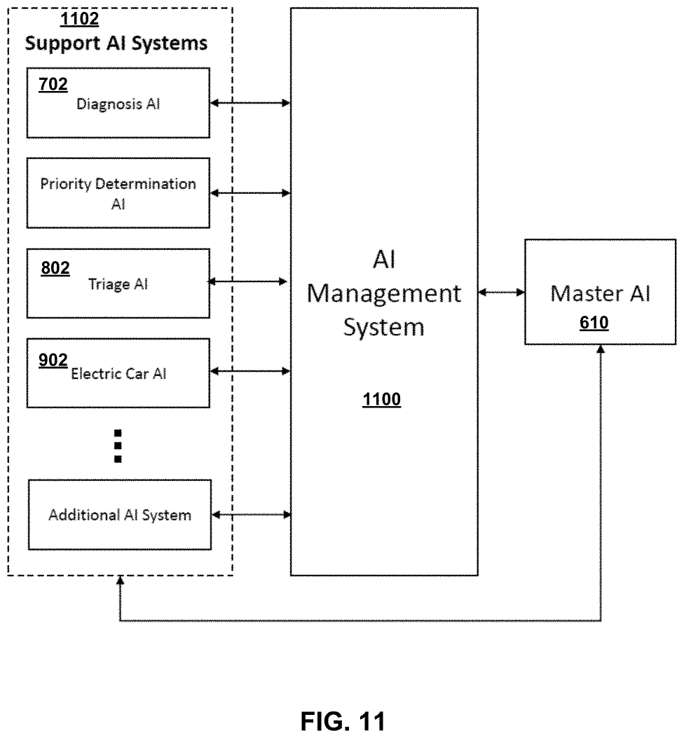

FIG. 11, illustrates, an example of an AI management system 1100, in accordance with some embodiments. This system 1100 may act as manager/trainer for all the AI used. Since the majority of AI relies on the data, the intelligent diagnosis system will learn on its own as it acquires more data. The re-learning or re-training will be performed by the AI management system 1100 with guidance from the health practitioners dentists, dental assistants, or even patients (i.e., supervised learning). The intelligent diagnosis system may also use other support AI systems to have added features. The AI management system 1100 ensures that the intelligent diagnosis system is interoperable (can work with other existing AI systems) or scalable (can accept other AI modules in the future).

In some embodiments, the initial data used to train the intelligent diagnosis system may only acquire a certain accuracy percentage or may not capture the full response of the system. This AI management system 1100 will provide a way of updating the support AI systems 1102 and the Master AI 610 to better serve the patients and dental practitioners.

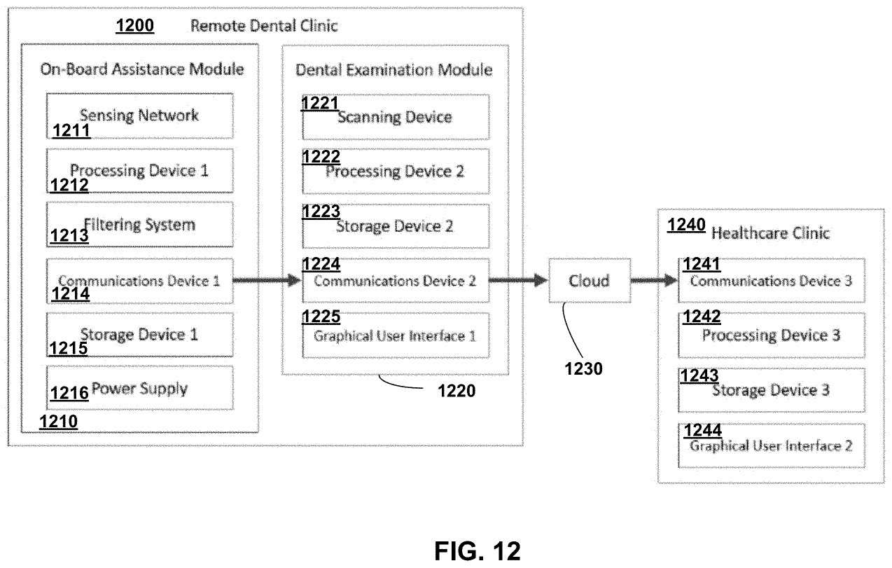

FIG. 12 illustrates an example of a remote dental clinic (or movable practice) 1200, in accordance with some embodiments. The remote dental clinic (or movable practice) 1200 comprises a vehicle preferably an electric-powered vehicle or more preferably a fully autonomous vehicle. It is preferable that the fully autonomous vehicle can be equipped with robotic diagnostic devices and/or artificial intelligence (AI) based diagnostic methods. The remote dental clinic 1200 comprises an on-board assistance module 1210 and a dental examination module 1220.

The electric vehicle may provide sufficient space for the service as it be providing all the features of an electric car. This electric vehicle will extend the four walls of the dental office as it will be mobile to any place. It has the benefits of giving service in all closed spaces like garages, underground parking spots, busy streets or even rural areas. Also, it has a capability to go autonomous.

The electric vehicle may be able to mitigate air pollutants as it has its own high-efficiency particulate air (HEPA) filter, inspired by filtration systems used in hospitals. This system will be able to strip the outside air pollen, bacteria, and pollution before they enter the cabin. In some embodiments, the design of the electric vehicle is minimal, as it will be able to hold the scanner and transform the passenger chair into a dental chair with its features. In addition, it is also economical and environmentally friendly.

In some embodiments, an on-board assistance module 1210 comprises a sensing network 1211, processing device 1 1212, filtering system 1213, communications device 1 1214, storage device 1 1215, and power supply 1216. The sensing network 1211 is a series of interconnected sensors that are strategically placed in predetermined locations in the remote dental clinic or in the electric-powered vehicle. The sensing network 1211 measures the quality of air inside the remote dental clinic and of the outside environment.

In some embodiments, the sensing network 1211 measures one or more patient's data such as but is not limited to the patient's health, temperature, blood pressure, heart rate, oxygen levels, emotions based on facial expressions, and level of satisfaction. Preferably, the sensing network 1211 also measures the environmental parameters such as but is not limited to the electric-powered vehicle's indoor temperature, humidity, air quality, and the electric-powered vehicle's surrounding environment's temperature, humidity, and air quality.

The sensing network 1211 sends the sensing data to a processing device 1 1212 for analysis and control of the filtering system and other actuators if necessary. The processing device 1 1212 also saves the sensing data to a storage device 1 1215 and converts the data into a format suitable for transmission or sending via a communications device 1 1214. In a preferred embodiment, the processing device 1 1212 is integrable with the electric-powered vehicles operating system. The processing device 1 1212 has limited access to the electric vehicle's operating system or core functionalities to ensure the safety in the electric vehicle's operation.

The filtering system 1213 is an air filtering device or more preferably a high-efficiency particulate air (HEPA) filter or a high-efficiency particulate arrestance filter for filtering pollen, bacteria, and pollution before entering the remote dental clinic.

The electric vehicle will be able to mitigate air pollutants as it has its own HEPA filter, inspired by filtration systems used in hospitals. This system will be able to strip the outside air pollen, bacteria, and pollution before they enter the cabin.

The communications device 1 1214 can be a wired or wireless communications device such as but is not limited to wireless fidelity (WiFi), bluetooth, LTE, 5G, etc.

The on-board assistance module's power supply 1216 can be a separate power storage device or the power source of the remote dental clinic or electric vehicle. The power supply 1216 uses a power management system for efficient distribution of power between the electric vehicle, the on-board assistance module, and the dental examination module. The power management system notifies the human operator of the current power level, remaining time to consume 80 to 100% of the power via an alert or notification module. In some embodiments, the power management system is connected to a human operator's mobile device or smartphone via a smartphone app for status checking and notification.

The dental examination module 1220 comprises a scanning device 1221, processing device 2 1222, storage device 1223, communications device 2 1224, and a graphical user interface 1 1225.

The scanning device 1221 is an optical device or a near infrared imaging (NRI) technology-based device for acquiring image data of a dental structure or the interior topographical features of the dental anatomy. The scanning device 1221 uses wavelength between approximately 250 nm hertz to approximately 1090 nm, or more preferably between approximately 500 nm to approximately 850 nm hertz excluding the harmful spectrums of light. It should be understood that other frequencies may be used. In some embodiments, the scanning device 1221 is a 3D mapping device that uses one or more stationary or movable optical devices for scanning the interior features or characteristics of the dental structure. The scanning device 1221 can scan the whole mouth environment even detect the interproximal caries lesions above the gingiva.

In some embodiments, the imaging device or a scanning device 1221 may provide accuracy and carry detection using NIRI (Near Infrared Imaging) technology. The imaging device may aid in detection and monitoring of interproximal caries lesions above the gingiva without using harmful radiation.

In some embodiments, the imaging device comprises an optical impression system used to record topographical images of the teeth and oral tissues. It captures 3D digital impressions of teeth, oral soft tissue and structures and bite relationships. The scanner produces a red laser light (e.g., 680 nm Class 1) as well as white LED emission.

The scanning device 1221 is connected to a processing device 2 1222. The processing device 2 1222 is an image processing device or an intelligent system for processing the data acquired reconstructs the image data acquired by the scanning device 1221 into a digital visual representation that can be understood by a human operator. The image data can then be digitally viewed in any angle and zoom level. The processing device 2 1222 uses an error detection and correction algorithm in the processing of the image data. In the event that the remote dental clinic is moving, the processing device 2 1222 performs an image stabilizing algorithm to correct errors due to the movement of the remote dental clinic or the abrupt movement of the patient during the scanning process. If the processing device 2 1222 identifies an error that exceeds the allowable threshold, the processing device 2 1222 notifies the human operator and suggests a rescanning step via a graphical user interface (GUI) 1 1225. The processing device 2 1222 then compares the first image with the second image and uses the data to improve the accuracy of the final image data. The processing device 2 1222 has a diagnostic tool for identifying any irregularities, disorders, diseases, or indicators of the health status of the dental anatomy. If the processing device 2 1222 identifies any irregularities, the processing device 2 1222 notifies the human operator, health practitioner, or patient via the graphical user interface (GUI) 1 1225. The notification may include suggestions of the procedures which may be appropriate to the patient such as but is not limited to oral prophylaxis, restoration, root canal, orthodontic treatment or a combination thereof.

The communications device 2 1224 is a wireless communications device such as but is not limited to wireless fidelity (WiFi), bluetooth, LTE, 5G, etc. The communications device 2 1224 sends the image data to a cloud service 1230 for storage and remote access. The cloud service 1230 is a subscription based secured database for storing the received image data. The cloud service 1230 can also be a website or webpage with a dedicated user interface which a human operator, health professional or patient can access securely and privately. The cloud service 1230 can be accessed by a healthcare clinic 1240 via a communications device 3 1241. It is conceivable that the healthcare clinic 1240 is a main hospital or health facility but can also be another remote dental clinic 1200. The communication between a remote dental clinic 1220, healthcare clinic 1240, or another remote dental clinic 1200 allows the exchange of data, medical opinions, and diagnostic tools which may be available to other remote dental clinics or in the main healthcare clinic which may have more advanced diagnostic tools or processing device 3 1241. The processing device 3 1241 can be a more complex computing device that may use more advanced intelligent systems or a large volume of patient data which may be stored, for example, in a storage device 3 1243. The processing device 3 1242 may use the large volume of patient data stored in the storage device 3 1243 to perform intelligent diagnostics with higher level of accuracy. A graphical user interface 2 1244 is also provided in the healthcare clinic to provide a visual presentation of the image data.

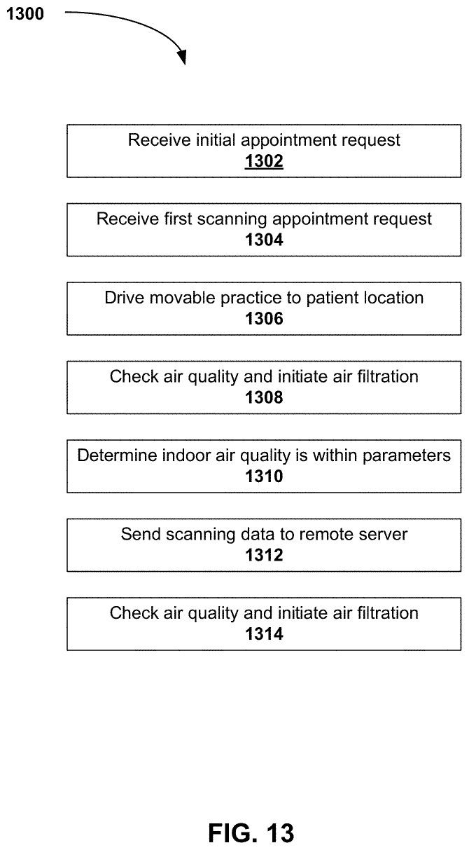

FIG. 13 illustrates, in a flowchart, an example of a method 1300 of data acquisition, in accordance with some embodiments.

In step 1302: Patients will reach out to an app or a website and book an initial appointment for dental diagnosis. The system will receive an initial appointment request.

In step 1304: From the app, a patient will be able to book their first scanning at their own convenience. The system will receive a first scanning appointment request.

In step 1306: Dental hygienist will be able to drive (or a self driving vehicle will drive the hygienist and) the “movable practice” to the requested time and site of the patient.

In step 1308: The sensing network (or the vehicle such as an electric vehicle) will check the indoor air quality. If the air quality is below the required level, air filtration will commence. Air filtration will continue until the air quality passes the required level.

In step 1310: If the air quality inside the vehicle (or hybrid or electric vehicle) is measured to be good or within the acceptable levels, a notification will be sent to the dental hygienist via the installed app. The notification will inform the dental hygienist and the patient that the dental procedure can be safely performed.

Prior to or upon arrival to the place, dental hygienist will be able to prepare and sanitize the chair and scanner. Once the patient is in the car, scanning will commence. Patients will be able to adjust the surroundings to achieve maximum comfort.

In step 1312: When the scanning is done, the scanning data may be sent to a remote server where it will be checked by a dentist remotely via iTero software and will be able to provide dental diagnosis and create a treatment plan promptly.

The dental team will reach out to the patient to schedule the first on-site visit/treatment. After the procedure, dental hygienists will perform sanitary methods to ensure utmost safety of the next patient.

In step 1314: The sensing network (or the vehicle) will check the indoor air quality. If the air quality is below the required level, air filtration will commence. Air filtration will continue until the air quality passes the required level. (The vehicle can now accommodate the next patient.)

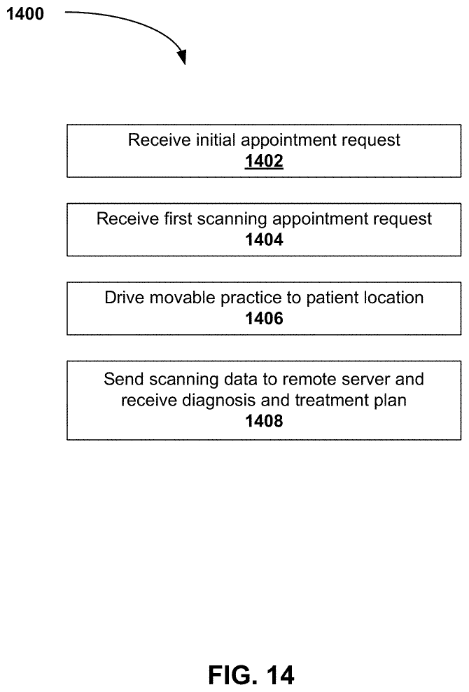

FIG. 14 illustrates, in a flowchart, an example of a method 1400 of diagnosis, in accordance with some embodiments.

In step 1402: Patients will reach out to an app or a website and book an initial appointment for dental diagnosis. The system will receive an initial appointment request.

In step 1404: From the app, patient will be able to book their first scanning at their own convenience. The system will receive a first scanning appointment request.

In step 1406: Dental hygienist will be able to drive (or a self driving vehicle will drive the hygienist and) the “movable practice” to the requested time and site of the patient

Upon arrival to the place, dental hygienist will be able to prepare and sanitize the chair and scanner. Once the patient is in the car, scanning will commence. Patients will be able to adjust the surroundings to achieve maximum comfort.

In step 1408: When the scanning is done, the scanning data may be sent to a remote server where it will be checked by a dentist remotely via iTero software and will be able to provide dental diagnosis and create a treatment plan promptly.

The dental team will reach out to the patient to schedule the first on-site visit/treatment. After the procedure, dental hygienists will perform sanitary methods to ensure utmost safety of the next patient.

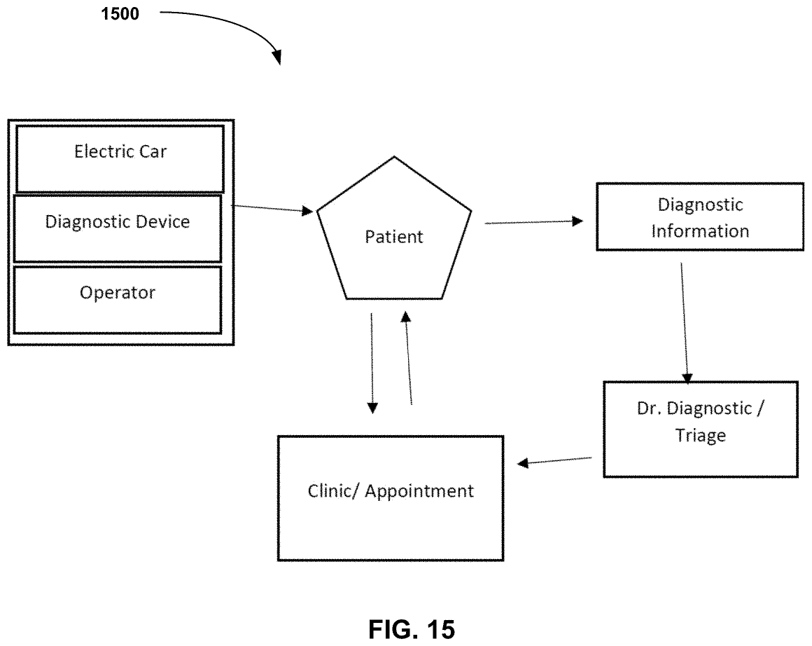

FIG. 15 illustrates an example of a model for an AI for diagnosis 1500, in accordance with some embodiments. In this scenario, once the system receives a patient request for an initial appointment, a movable practice is sent to the patient's location. The movable practice pertains to the electric car with the operator having the diagnostic device. The operator then attends to the patient's request and receives the scanning data of the patient via the device. The operator (via the application on a device) or the system may then suggest the patient to visit a clinic or a preferred appointment location for further oral examination or oral procedure. The patient (via the application on a device) or system can also send the scanning data or check up data to a remote clinic to process the diagnosis information. The processed diagnostic application can then be sent to another diagnostic clinic, doctor, or health specialist for further review and triage. Based on the triage, the patient's appointment will be prioritized, scheduled, and set.

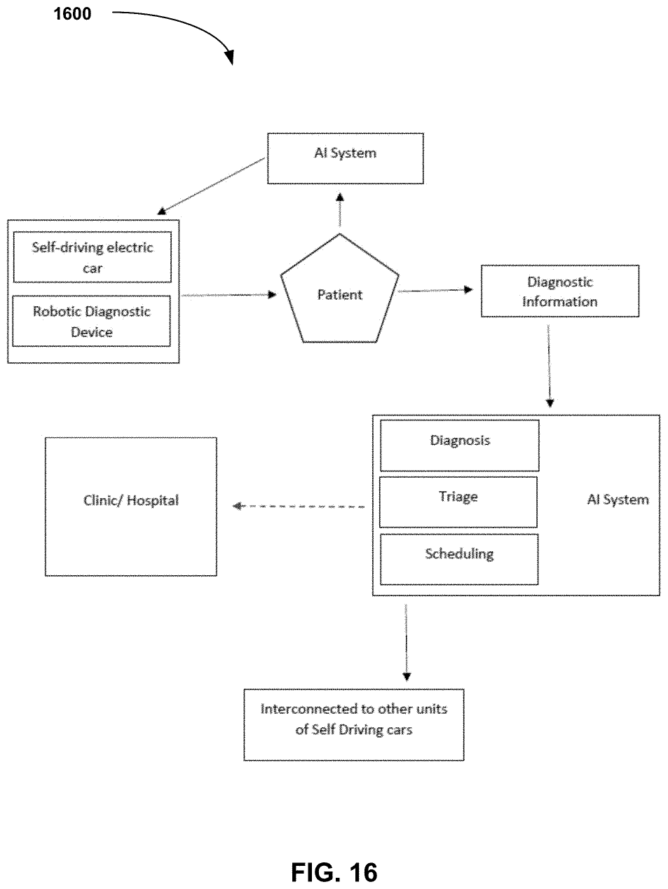

FIG. 16 illustrates another example of a model for an AI for diagnosis 1600, in accordance with some embodiments. In this scenario, the patient (via an application on a device) sends a request for an initial appointment to an AI system having a scheduling and prioritization function. The AI system appoints a movable practice, being the self-driving electric vehicle with the robotic diagnostic device, to attend the patient. The movable practice acquires the scanning data of the patient and processes the data to have an initial diagnostic information. In some embodiments, the patient (via the application on a device) or the system also sends the diagnostic information to another AI system that can perform further diagnosis, triage, and/or scheduling. The said AI system can then recommend to the patient to proceed to a clinic or hospital based on the performed diagnosis or triage. In another embodiment, one or more AI systems are interconnected to other units of movable practice or self-driving cars.

In some embodiments, the self-driving electric vehicle may include an air filtration and sanitation system and a safety system. The air filtration and sanitation system may identify air quality, purify the air, and comprise a UVC light cleaner. The safety system may provide external screening for a patient, and chair safety.

In some embodiments, the robotic diagnostic device may perform a patient scan and may comprise a programmable diagnostic tool. The patient scan may be intra-oral, extra-oral or a CBCT-CT scan. The programmable diagnostic tool may differ depending upon specialty needed. For example, for the dental field, a heat test and a percussion test may be performed by the diagnostic tool.

Table 1 illustrates possible outcomes for examples of uses of the AI for diagnosis system.

TABLE 1
AI for Diagnosis

| | Example 1 | Example 2 | Example 3 |
|---|---|---|---|
| Diagnosis | Gingivitis | Pulpitis | PT may be having MI. |
| Triage | Non-Urgent | Urgent | Take PT to the |
| Scheduling | Cleaning | Offer closest | closest hospital or |
| | Session | RCT session | urgent care. |

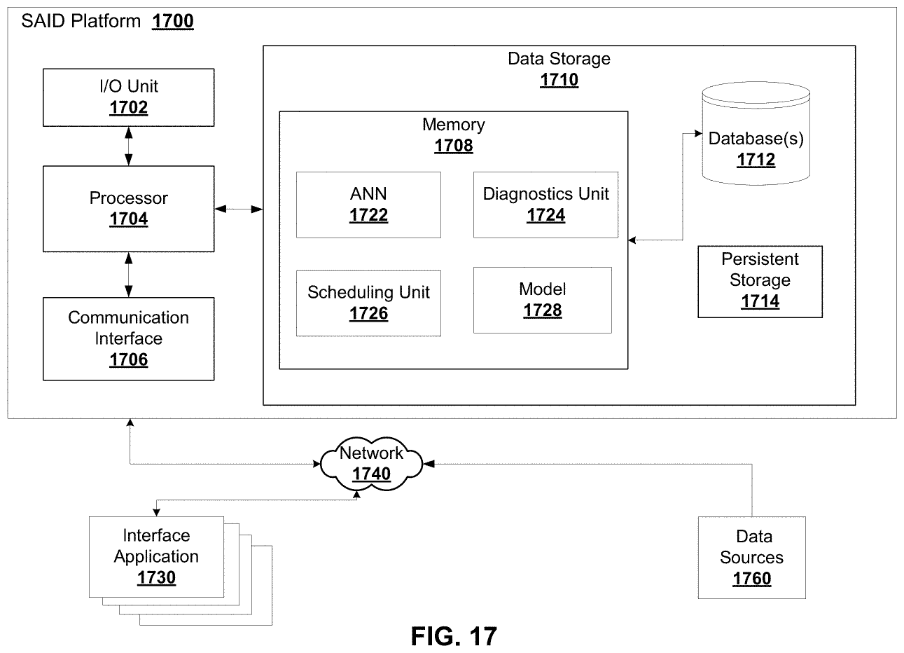

FIG. 17 illustrates, in a schematic diagram, an example of a machine learning prediction platform 1700, in accordance with some embodiments. The platform 1700 may be an electronic device connected to interface application 1730 and data sources 1760 via network 1740. The platform 1700 can implement aspects of the processes described herein.

The platform 1700 may include a processor 1704 and a memory 1708 storing machine executable instructions to configure the processor 1704 to receive a voice and/or text files (e.g., from I/O unit 1702 or from data sources 1760). The platform 1700 can include an I/O Unit 1702, communication interface 1706, and data storage 1710. The processor 1704 can execute instructions in memory 1708 to implement aspects of processes described herein.

The platform 1700 may be implemented on an electronic device and can include an I/O unit 1702, a processor 1704, a communication interface 1706, and a data storage 1710. The platform 1700 can connect with one or more interface applications 1730 or data sources 1760. This connection may be over a network 1740 (or multiple networks). The platform 1700 may receive and transmit data from one or more of these via I/O unit 1702. When data is received, I/O unit 1702 transmits the data to processor 1704.

The I/O unit 1702 can enable the platform 1700 to interconnect with one or more input devices, such as a keyboard, mouse, camera, touch screen and a microphone, and/or with one or more output devices such as a display screen and a speaker.

The processor 1704 can be, for example, any type of general-purpose microprocessor or microcontroller, a digital signal processing (DSP) processor, an integrated circuit, a field programmable gate array (FPGA), a reconfigurable processor, or any combination thereof.

The data storage 1710 can include memory 1708, database(s) 1712 and persistent storage 1714. Memory 1708 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Data storage devices 1710 can include memory 1708, databases 1712 (e.g., graph database), and persistent storage 1714.

The communication interface 1706 can enable the platform 1700 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these.

The platform 1700 can be operable to register and authenticate users (using a login, unique identifier, and password for example) prior to providing access to applications, a local network, network resources, other networks and network security devices. The platform 1700 can connect to different machines or entities.

The data storage 1710 may be configured to store information associated with or created by the platform 1700. Storage 1710 and/or persistent storage 1714 may be provided using various types of storage technologies, such as solid state drives, hard disk drives, flash memory, and may be stored in various formats, such as relational databases, non-relational databases, flat files, spreadsheets, extended markup files, etc.

The memory 1708 may include an ANN 1722, an diagnostics unit 1724, a scheduling unit 1726, and a model 1728.

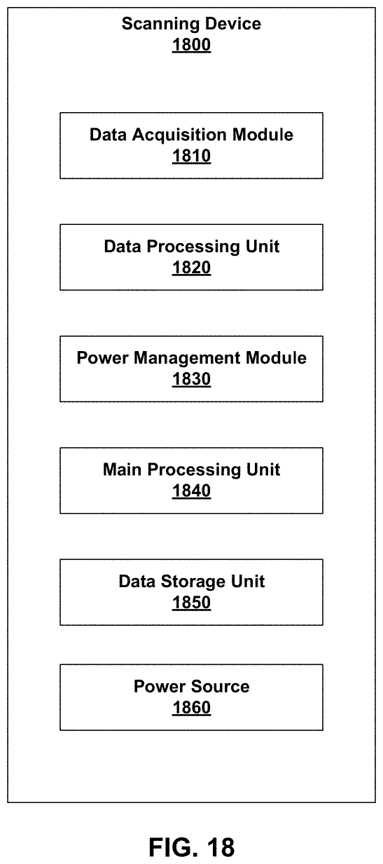

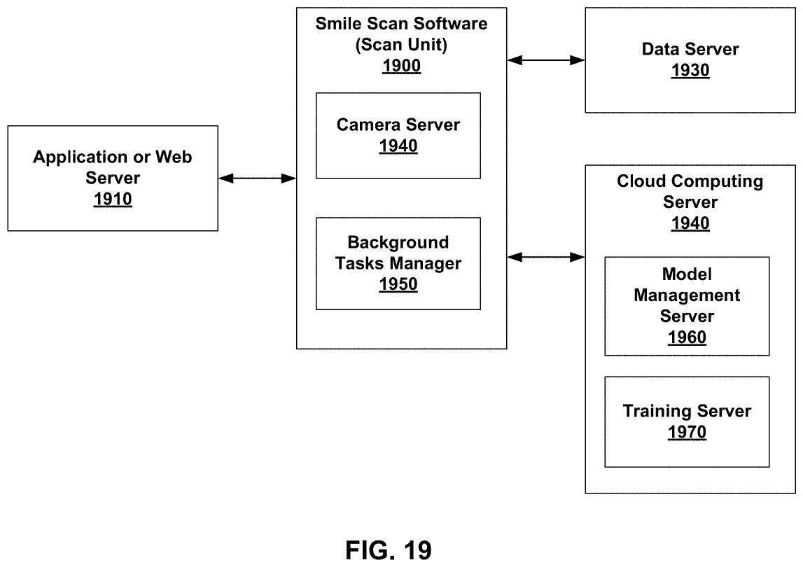

Various examples will now be described more fully with reference to the accompanying drawings in which some examples are illustrated. In the figures, the thicknesses of lines, layers and/or regions may be exaggerated for clarity. In some embodiments, FIGS. 18 and 19 below expand upon the system and scanning device as described above with reference to FIG. 12.

FIG. 18 is a schematic diagram of a scanning device 1800, in accordance with some embodiments. The scanning device 1800 may be a handheld medical device used for checking, evaluating, or examining a patient's oral health. The scanning device 1900 comprises a data acquisition module 1810, data processing unit 1820, power management module 1830, main processing unit 1840, data storage unit 1850, and a power source 1860.

The data acquisition module 1810 is preferably an optical scanning device or a near infrared imaging (NRI) technology-based device for acquiring image or video data of the oral cavity, dental structure, or the interior topographical features of the dental anatomy. In a preferred embodiment, the data acquisition module 1810 may be any device capable of acquiring at least one oral feature which can be impressions of the teeth, gingiva, oral soft tissues, bite relationships, tongue, surfaces of the oral cavity, or combinations thereof. The image, video data, or oral feature that is acquired by the data acquisition module 1810 are transferred to the data processing unit 1820. The data processing unit 1820 configures the data acquisition module 1810 and preprocesses the acquired data image or video data. After preprocessing, the data are sent to the main processing unit 1840 for further processing and analysis. The main processing unit 1840 stores the received data in a data storage unit 1850. The main processing unit 1840 also manages the efficient power distribution in the scanning device 1800 via a power management module 1830. The power management module 1830 is a power supplying device that regulates the power received from a power source 1860. The regulated power is then distributed to the other modules or units of the scanning device 1800 based on the required power levels.

In one embodiment, the data processing unit 1820 can be any microcontroller, microprocessor, central processing unit (CPU), graphics processing unit (GPU), tensor processing unit (TPU), field programmable gate arrays (FPGA), or any hardware device capable of processing data, issuing instructions, or executing calculations based on the data provided by the data acquisition module 1810.

In another embodiment, the power management module 1830 is a device capable of balancing the load of the scanning device 1800 by ensuring that the correct voltages and ampere ratings are provided for each of the scanning device's 1800 components such as the data acquisition module 1810, data processing unit 1820, main processing unit 1840, data storage unit 1850, and a power source 1860. In a preferred embodiment, the power management module 1830 can be a smart load protection device designed to protect the scanning device 1800 from damage caused by an overload condition or short circuit. The power management module 1830 detects a fault condition and interrupts current flow to the main processing unit 1840.

In yet another embodiment, the main processing unit 1840 can be any microcontroller, microprocessor, central processing unit (CPU), graphics processing unit (GPU), tensor processing unit (TPU), field programmable gate arrays (FPGA), or any hardware device capable of processing data, issuing instructions, or executing calculations. Preferably, the main processing unit 1840 can use advanced processing means such as artificial intelligence (AI), intelligent systems, predictive algorithm, artificial neural networks (ANN), fuzzy logic, genetic algorithm (GA), machine learning (ML), deep learning, or combinations thereof. The main processing unit 1840 is connected to a data storage unit 1850 or to an internal or external memory device.

In still another embodiment, the data storage unit 1850 can be any medium or mechanism for storing or transmitting information in a form readable by a machine or computer. The memory device can have a primary memory device and/or a secondary memory device as a backup storage device. The memory device can be a read only memory (ROM), random access memory (RAM), magnetic disk storage media, hard disk storage, optical storage media, flash memory devices, universal serial bus (USB) drive, secure digital (SD) card, memory chip, or a combination thereof.

The power source 1860 may be any energy storage device such as one or more batteries.

The scanning device 1800 and its components are preferably enclosed in a housing or chassis having a main frame for holding the data acquisition module 1810, data processing unit 1820, power management module 1830, main processing unit 1840, data storage unit 1850, and the power source 1860. The data acquisition module 1810 can be enclosed in a dedicated housing that protects the sensitive optical scanning device. The dedicated housing is then coupled to the main frame. A top cover and a bottom cover are also provided to further protect the main frame in such a manner that the main frame is sandwiched by the said top and bottom covers.

In one embodiment, the data acquisition module 1810 comprises a three-dimensional mapping device having one or more stationary or movable optical devices for scanning the interior features or characteristics of an oral cavity or a dental structure. For example, the data acquisition module 1810 can scan the whole oral cavity or interior mouth environment to detect abnormalities such as interproximal caries lesions above the gingiva.

FIG. 19 illustrates, in a block diagram, the network connection of the software and processing units of the system, in accordance with some embodiments. The Smile Scan software (i.e., scan unit) 1900 may comprise an application, software, graphical user interface (GUI) or an interface installed in a scanning device 1800. This can be used by a user or a medical practitioner to access or operate a scanning device 1800. The scan unit 1900 comprises a camera server 1940 and a background tasks manager 1950. The scan unit 1900 can access an application or a web server 1910 for remote control and operation. For example, the scan unit 1900 can send real time data while a user is using the scanning device 1800. In this way, the medical practitioner can remotely guide the user while operating the scanning device 1800. Likewise, the scan unit 1900 can establish a communication with one or more cloud computing servers 1920 if the scan unit 1900 needs supplementary processing power. The scan unit 1900 can also directly access a data server 1930 directly or via the cloud computing server 1920. The cloud computing server 1920 comprises one or more model management servers 1960 and one or more training servers 1970.

The Smile Scan software or a scan software (i.e., scan unit) 1900 comprises a camera server 1940 and a background tasks manager 1950. The camera server 1940 is configured to operate the data acquisition module 1810 to acquire image or video data, generate metadata for the acquired image or video data, and save the image or video data with the metadata in the data storage unit 1850. The camera server 1940 can send or transmit the image or video data after acquisition. The background tasks manager 1950 can perform tasks such as periodically checking for new saved data on the data storage unit 1850; syncing new data with a remote data server, web server 1910 or cloud computing server 1920; deleting synced data from the data storage unit 1850; and powering off the scanning device 1800 when the tasks are completed.

The application or web server 1910 provides an interface to the patient or the medical practitioner. The application or web server 1910 can be a user mobile application that is configured to have one or more abilities or features such as but is not limited to: setting up, configuring, and sending commands to the scanning device 1800; communicating with the scan unit 1900; setting up the network; providing user setup; setting the capture parameters; initiating the capture command; managing the captured images; managing the user tooth health status; getting messages and alert notifications; managing subscriptions; getting healthy service; supporting patients or customers via voice or text; and getting consultation from dentists or dental practitioners via voice or text.

In another embodiment, the application or web server 1910 can be a dentist panel or a software providing an interface to the dentist or dental practitioner. The dentist panel can be used for answering patient inquiries; managing services, orders, and finances; and providing feedback or training data to the cloud computing server 1920 having the model management servers 1960 and the training server 1970. The process of providing feedback or training data to the cloud computing server 1920 can help the model management servers 1960 and the training server 1970 improve the AI models through reinforcement learning. The feedback or training data that can be provided to the server 1920 can be related to automatic speech recognition (ASR), natural language processing (NLP), text-to-speech synthesis (TTS), teeth detection, and teeth issue detection.

The cloud computing server 1920 can be one or more remotely available complex processing units which can be servers, databases, computers, microcontrollers, microprocessors, or any hardware device capable of processing data, issuing instructions, or executing calculations wherein the processing units can effectively communicate with each other. In some embodiments, the cloud computing server 1920 can perform parallel computing if complex data, analysis, or decision is required. If the cloud computing server 1920 identifies an error, then the cloud computing server 1920 can roll back the changes, restart the applications that fail, or self-heal wherein the cloud computing server 1920 automatically repairs or remedies the errors.

Herein, the cloud computing server 1920 is a modular system that can use or combine different applications, clusters, nodes, or a plurality of processing modules depending on the desired service or process. In the preferred embodiment, the one or more processing modules can perform simple to advanced processing means such as artificial intelligence (AI), intelligent systems, predictive algorithm, artificial neural networks (ANN), fuzzy logic, genetic algorithm (GA), machine learning (ML), deep learning, or combinations thereof. The cloud computing server 1920 can monitor the health of the different applications, clusters, nodes, or a plurality of processing modules used in the system. It is also conceivable that the one or more processing modules can function independently or may work synergistically based on the desired process or service for the user. The one or more processing modules can be replicated or duplicated which means that a processing module can have one or more replicas. The cloud computing server 1920 can identify an error in one or more processing modules. If an error is identified, then the cloud computing server 1920 can replace the said processing module with a replica that has the same functions of the said processing module. In this way, even if the system encounters an error, the cloud computing server 1920 can efficiently “self-heal” or repair itself ensuring a continuity of service.

In another embodiment, the cloud computing server's 1920 processing modules may comprise of model management servers 1960 and one or more training servers 1970. The functions of either or both the model management servers 1960 and training servers 1970 can be performed by or integrated to the cloud computing server 1920. The cloud computing server's 1920 processing modules may further comprise a teeth detection module, and a teeth recognition module.

The model management servers 1960 manages and monitors the performance of different AI or ML models. The model management server 1960 can be a processing unit or device employing a platform for managing the machine learning (ML) lifecycle which includes the experimentation, reproducibility, deployment, and a central model registry. The AI or ML models can pertain to the dental or health models indicative of healthy oral parameters. In a preferred embodiment, the model management server 1960 further comprises a tracking component, projects component, models component, and a registry component. The tracking component is configured to log parameters, code versions, metrics, and other related data. The projects component is for packaging the code for execution. The models component deploys machine learning models. The registry component stores, annotates, and further manages the models in a database.

The training server 1970 is configured for creating AI models and for distributed training of the models. The training server 1970 also curates training data and trains large-scale models. The training server 1970 comprises one or more processing modules or models such as automatic speech recognition (ASR) module, natural language processing (NLP) module, text-to-speech synthesis (TTS) module, teeth detection module, and teeth recognition module. The natural language processing (NLP) module determines and uses punctuations to normalize text format before saving in context, text classification for classifying texts in automatic speech recognition (ASR) module, and for question answering based on context. The automatic speech recognition (ASR) module provides speech to text services wherein the person's speech is processed and converted into text data. In some cases, the ASR determines the customer language and translates the language into a more understandable language. The text-to-speech synthesis (TTS) module coverts text data into a speech which can either be a male or female voice. The language can also be translated based on the customer's language.

In another embodiment, the training server 1970 can train recognition models based on a speaker or person's identity and teeth models. The training server 1970 can also fine tune the automatic speech recognition (ASR) module, natural language processing (NLP) module, text-to-speech synthesis (TTS) module, teeth detection module, and teeth recognition module.

In yet another embodiment, the processing modules that can be used by the cloud computing server 1920 comprises an audio/video analyzer module, sound recognition module, speaker recognition module, context maker module, command queue manager module, command runner module. The audio/video analyzer module exports metadata from the received audio or video data. The audio/video analyzer module can also classify if the audio or video data are for or from humans or objects. The sound recognition module classifies audio data, interprets the audio data, and generates one or more labels for each audio data. The speaker recognition module determines a person's identity based on the received audio data and the saved audio data. If the speaker recognition module fails to determine or recognize the audio data, then the speaker recognition module acquires the metadata and trains the AI models to recognize the person's identity in the future. The context maker module acquires and analyzes the data from the natural language processing (NLP) module. The context maker module then defines the type of text or data, saves, and updates a customer context. The context maker module saves or stores every data related to the customers with different format, preferably in a high-performance database. The data related to the customers can be the identity of the customer, reminders, OCR results, health records, image data, or audio and video data. The context maker module also generates commands based on generated texts and sends or saves the commands on the command queue manager module. The command queue manager module manages the commands received from the context maker module or all throughout the cloud computing server 1920. The command runner module acquires one or more commands from the command queue manager module, analyzes the acquired commands, and executes the commands with metadata.

In an embodiment, the processing modules that can be used by the cloud computing server 1920 comprises video core feature module, skills module, and contribution module. The video core feature module can perform teeth recognition, issue recognition, and text on wild detection. The skills module can process one or more reminders, optical character recognition or optical character reader (OCR). The skills module can also perform web searching. The contribution module acquires additional data, feedback, contributions for improving the cloud computing server 1920 or the system for intelligent diagnosis. The contribution module may use the acquired data to optimize the automatic speech recognition (ASR) module, natural language processing (NLP) module, text-to-speech synthesis (TTS) module, teeth detection module, teeth recognition module, optical character recognition or optical character reader (OCR), and other processing modules that can be added to the system.

In some embodiments, the cloud computing server 1920 can effectively select and combine one of the above processing modules to perform the operations relating to the system and method for intelligent diagnosis.

The data server 1930 can be one or more databases, local database, remote database, data lakes, cloud storage, or other means of storing data. The data server 1930 is configured to receive image or video data having metadata; perform processing on the received data such as breaking down video frames into individual frames; store data to the database; update the database; and communicate with the cloud computing server 1920 having the model management servers 1960 and the training server 1970. Preferably, the one or more data servers 1930 are connected to the scan software 1900 and to the cloud computing server 1920; wherein the one or more data servers 1930 can send and store data to and from the scan software 1900 and the cloud computing server 1920.

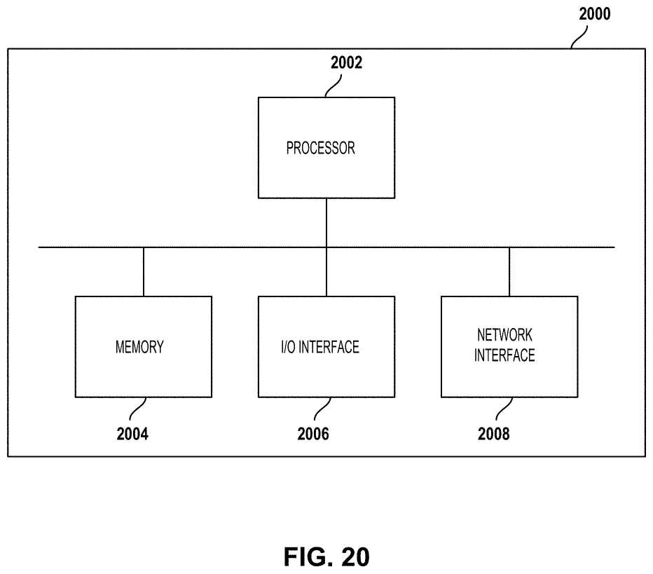

FIG. 20 is a schematic diagram of a computing device 2000 such as a server or other computer for processing, control, interface, or monitoring in a device. As depicted, the computing device includes at least one processor 2002, memory 2004, at least one I/O interface 2006, and at least one network interface 2008.

Processor 2002 may be an Intel or AMD x86 or x64, PowerPC, ARM processor, GPU, DSP, FPGA, CPLD, or the like. Memory 2004 may include a suitable combination of computer memory that is located either internally or externally such as, for example, random-access memory (RAM), read-only memory (ROM), compact disc read-only memory (CDROM).

Each I/O interface 2006 enables computing device 2000 to interconnect with one or more input devices, such as a keyboard, mouse, camera, touch screen and a microphone, or with one or more output devices such as a display screen and a speaker.

Each network interface 2008 enables computing device 2000 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX, 5G), SS7 signaling network, fixed line, local area network, wide area network, and others.

The foregoing discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. A system for intelligent diagnosis, the system comprising:
at least one processor; and
a memory comprising instructions which, when executed by the processor, configure the processor to:
receive an initial appoint request;
receive a first scanning appointment request;
send a movable practice to patient location;
receive scanning data; and
send the scanning data to remote server for processing.

2. The system as claimed in claim 1, wherein the processor is configured to at least one of:
check air quality in the movable practice; or
initiate air filtration if the air quality is below a threshold.

3. The system as claimed in claim 1, wherein the processor is configured to schedule a follow-up appointment based on the scanning data.

4. The system as claimed in claim 3, wherein the processor is configured to determine a priority score based on the scanning data and triage data.

5. The system as claimed in claim 4, wherein the processor is configured to determine an appointment time based on the priority score and a movable practice availability.

6. The system as claimed in claim 5, wherein the processor is configured to determine the movable practice availability based on logistics data.

7. A method of intelligent diagnosis, the method comprising:
receiving an initial appoint request;
receiving a first scanning appointment request;
sending a movable practice to patient location;
receiving scanning data; and
sending the scanning data to remote server for processing.

8. The method as claimed in claim 7, wherein the processor is configured to at least one of:
check air quality in the movable practice; or
initiate air filtration if the air quality is below a threshold.

9. The method as claimed in claim 7, wherein the processor is configured to schedule a follow-up appointment based on the scanning data.

10. The method as claimed in claim 9, wherein the processor is configured to determine a priority score based on the scanning data and triage data.

11. The method as claimed in claim 10, wherein the processor is configured to determine an appointment time based on the priority score and a movable practice availability.

12. The method as claimed in claim 11, wherein the processor is configured to determine the movable practice availability based on logistics data.

13. A system for intelligent diagnosis, comprising:
a scan unit having a camera server and a background tasks manager;
one or more application server in communication with the scan unit;
a cloud computing server, connected to the scan unit and the data server, said cloud computing server configured to use one or more processing modules; and
one or more data servers, connected to the scan unit and to the cloud computing server;
wherein the one or more data servers send and store data to and from the scan unit and the cloud computing server.

14. The system of claim 13, wherein the cloud computing server comprises at least one of:
a model management server and a training server;
a teeth detection module, and a teeth recognition module;
an audio/video analyzer module, sound recognition module, speaker recognition module, context maker module, command queue manager module, command runner module; or
a video core feature module, skills module, and contribution module.

15. The system of claim 14, wherein the training server comprises an automatic speech recognition (ASR) module, natural language processing (NLP) module, text-to-speech synthesis (TTS) module, teeth detection module, and teeth recognition module.

16. The system of claim 13, wherein the cloud computing server is configured to identify an error in one or more processing modules and replace the said processing module with a replica that has the same functions of the said processing module.

17. A scanning device for intelligent diagnosis, comprising:
a data acquisition module for acquiring at least one oral feature;
a main processing unit coupled to the data acquisition module and configured to save the at least one oral feature to a data storage unit;
a power source for supplying power to the data acquisition module and the main processing unit; and
an interface configured to accept one or more triggering actions from a user.

18. The scanning device of claim 17, comprising at least one of:
a data processing unit coupled to the data acquisition module and configured to operate the data acquisition module;
a power management module for regulating the supplied power to the main processing unit and the data acquisition module; or
a communications module for sending and receiving data from another scanning device or a remote processing device.

19. The scanning device of claim 17, wherein the oral feature comprises impressions of the teeth, gingiva, oral soft tissues, bite relationships, tongue, surfaces of the oral cavity, or combinations thereof.
